(19) United States
(12) Patent Application Publication
(10) Pub. No.: US 2009/0005788 A1
(43) Pub. Date: Jan. 1, 2009

(54) Intraocular Lens Injector

(76) Inventor: Brian D. Rathert, Largo, FL (US)

Correspondence Address: Bausch & Lomb Incorporated, One Bausch & Lomb Place, Rochester, NY, 14604-2701, US

(21) Appl. No.: 11/768,530

(22) Filed: Jun. 26, 2007

(51) Int. Cl. A61F 9/007

(52) U.S. Cl. 606/107

(57) ABSTRACT

An IOL injector, comprising an injector body having a lumen wall defining a lumen, a plunger configured and arranged to move through the lumen, and a sleeve, the sleeve configured and arranged so as to contact at least a portion of the lumen wall and to slidably engage with at least a portion of the plunger.

### FIELD OF THE INVENTION

The present invention relates to intraocular lens (IOL) injectors, and more particularly to IOL injectors providing plunger control.

### BACKGROUND OF THE INVENTION

IOLs are artificial lenses used to replace or supplement the natural crystalline lenses of patients' when their natural lenses are diseased or otherwise impaired. IOLs may be placed in either the posterior chamber or the anterior chamber of an eye. IOLs come in a variety of configurations and materials.

Various instruments and methods for implanting such IOLs in an eye are known. Typically, an incision is made in a patient's cornea and an IOL is inserted into the eye through the incision. In one technique, a surgeon uses surgical forceps to grasp the IOL and insert it through the incision into the eye. While this technique is still practiced today, more and more surgeons are using IOL injectors, which offer advantages such as affording a surgeon more control when inserting an IOL into an eye and permitting insertion of IOLs through smaller incisions. Relatively small incision sizes (e.g., less than about 3 mm) are preferred over relatively large incisions (e.g., about 3.2 to 5+mm) since smaller incisions have been attributed with reduced post-surgical healing time and reduced complications such as induced astigmatism.

In order for an IOL to fit through a small incision, it is typically folded and/or compressed by passing the IOL through a nozzle providing a funnel-shaped portion of the injector lumen. After the IOL exits the distal end of the nozzle and enters the eye, it assumes its original unfolded/uncompressed shape.

FIG. 1A is a schematic illustration of a conventional IOL injector 10. FIG. 1B is a cut away cross section of a portion of the injector of FIG. 1A. Injector 10 comprises an injector body 20, a nozzle 60, and a plunger 30 that is substantially fully retracted within the injector body (i.e., the plunger is substantially fully withdrawn from the injector body). The plunger comprises a plunger body 32 and a plunger shaft 34. The distance traveled by the plunger between full retraction and full extension (i.e., where the plunger is actuated far enough to fully deliver the IOL into an eye) is called the plunger stroke S. In injector 10, full extension occurs when the face 35 of the plunger body contacts surface 25 of the injector body.

Stability of the plunger 30 within the injector body is desirable to provide a user with a robust feeling when injecting an IOL, and to help ensure proper engagement of plunger tip 40 with IOL 50. Plunger stability may be particularly troublesome for molded plastic injectors, but also occurs in other injectors.

Further, a plunger may be particularly unstable at points of the stroke near full retraction, where interface I between the plunger and the injector body is relatively small.

To increase the interface I between the injector body and the plunger, the injector body may be made longer or the stroke may be reduced. However, in at least some instances, increasing injector length is undesirable and may make the injector more cumbersome to handle, and reduced stroke is undesirable because it reduces control of the injector, for example, by reducing the distance over which compression of the IOL occurs.

A further contributor to plunger instability is the length of cantilever C of the plunger shaft. A long cantilever may increase the likelihood of bending or warping while the inserter is on a shelf prior to use. A long cantilever may also exacerbate bending of a plunger shaft that may occur as force is applied to an IOL when the IOL is injected into an eye. Bending may be particularly apparent while the lens traverses a funnel-shaped portion of the lumen and is being compressed, such bending may increase the likelihood of damage to the IOL during delivery.

### SUMMARY

Aspects of the present invention are directed to an injector including a sleeve to increase stability of the plunger.

An aspect of the invention is directed to an IOL injector, comprising an injector body having a lumen wall defining a lumen, a plunger configured and arranged to move through the lumen, and a sleeve, the sleeve configured and arranged so as to contact at least a portion of the lumen wall and to slidably engage with at least a portion of the plunger.

In some embodiments, the sleeve is configured and arranged so as to slidably engage the lumen wall. The sleeve may comprise a cylindrically-shaped tube. The sleeve may comprise an end cap configured and arranged to support a shaft of the plunger.

In some embodiments, the IOL injector comprises a first spring disposed between the sleeve and the injector body, and a second spring disposed between the plunger body and the sleeve. The sleeve may be adapted to be maintained in a fixed location within the injector body.

The sleeve may comprise a different material than at least one of the plunger and the injector body. In some embodiments, the sleeve comprises a different material than the plunger (i.e., the sleeve may or not be the same as the material of the injector body). In some embodiments, the sleeve comprises a different material than the injector body (i.e., the sleeve may or not be the same as the material of the plunger).

### BRIEF DESCRIPTION OF THE DRAWINGS

Illustrative, non-limiting embodiments of the present invention will be described by way of example with reference to the accompanying drawings, in which the same reference number is used to designate the same or similar components in different figures, and in which:

FIGS. 1A and 1B illustrate a projection view and a cross sectional view of a conventional injector, respectively;

FIGS. 2A and 2B illustrate, respectively, a projection view and a cross sectional view of an example of embodiment of an IOL injector according to aspects of the invention;

FIGS. 3A and 3B illustrate, respectively, a plan view and a projection view of the example of a sleeve shown in FIGS. 2A and 2B;

FIGS. 4A and 4B illustrate, respectively, a projection view and a cross sectional view of another example of embodiment of an IOL injector according to aspects of the invention;

FIGS. 5A and 5B illustrate, respectively, a plan view and a projection view of the example of a sleeve shown in FIGS. 4A and 4B; and

FIGS. 6A and 6B illustrate, respectively, a projection view and a cross sectional view of another example of embodiment of an IOL injector according to aspects of the invention.

### DETAILED DESCRIPTION

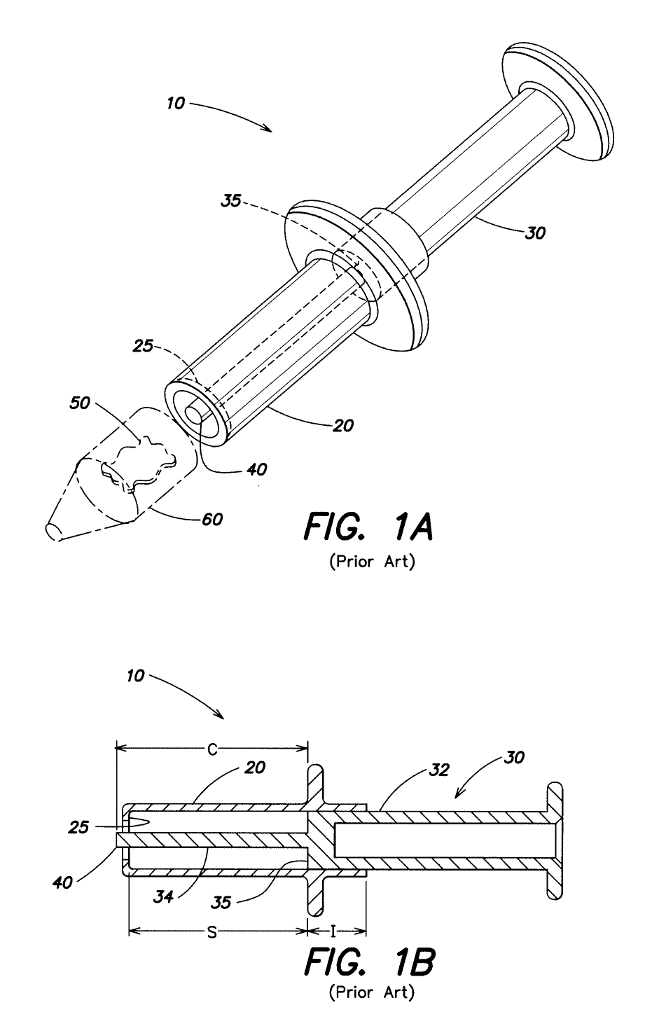

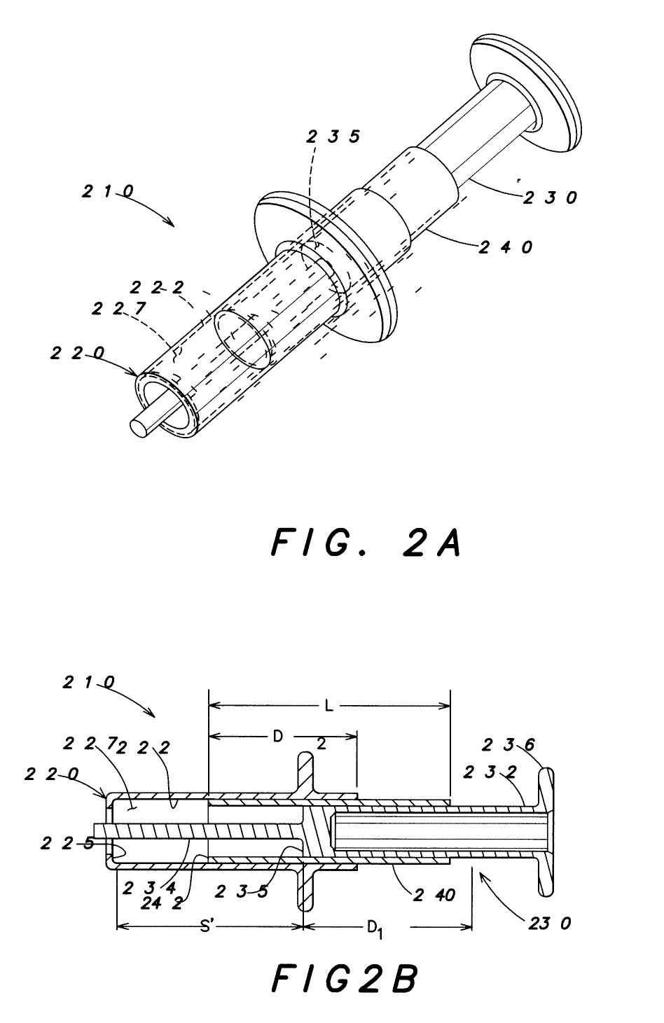

FIG. 2A is a schematic view of an example embodiment of an IOL injector 210 according to aspects of the invention; and FIG. 2B is a cross sectional view of the embodiment of an IOL injector. IOL injector 210 includes an injector body 220, a plunger 230, and a sleeve 240 having a length L. Sleeve 240 is disposed intermediate at least a portion of the plunger and at least a portion of the injector body. Plunger 230 is shown at full retraction. The nozzle and the IOL (as shown in FIG. 1A and 1B) are omitted from FIGS. 2A and 2B and from the FIGs. discussed below to avoid obfuscation. It is to be understood that an injector as shown in any of the FIGs. may include a nozzle of any suitable configuration, the nozzle having a hole for delivery of an IOL into an eye.

Injector body 220 has a lumen wall 222 defining a lumen 227. Plunger 230 is configured and arranged to move through the lumen. The wall may completely enclose the lumen along the lumen's entire length or have one or more openings (not shown). The lumen need only be sufficient to conduct the plunger along a suitable stroke (e.g., stroke S′). For illustrative purposes, injector body 220 and plunger 230 are illustrated with the same size and shape as injector body 20 and plunger 30 of prior art injector 10 in FIG. 1 so that strokes S and S′ are equal. Although the plunger and lumen are illustrated as having circular cross sections, any suitable cross sectional shape may be used.

Sleeve 240 is configured and arranged so as to contact at least a portion of the lumen wall 222 and to slidably engage with at least a portion of the plunger 230. In the illustrated embodiment, sleeve 240 slidably engages lumen wall 222. As illustrated in FIG. 2B, plunger 230 contacts sleeve 240 over a distance D₁ and sleeve 240 contacts the injector body over a distance D₂. In the illustrated embodiment, both D₁ and D₂ are greater than interface I (shown in FIG. 1) despite the fact that overlap between the body 220 and plunger 230 is the same as interface I in the injector shown in FIGS. 1A and 1B; however, such a configuration is not necessary to achieve advantages in accordance with aspects of the present invention.

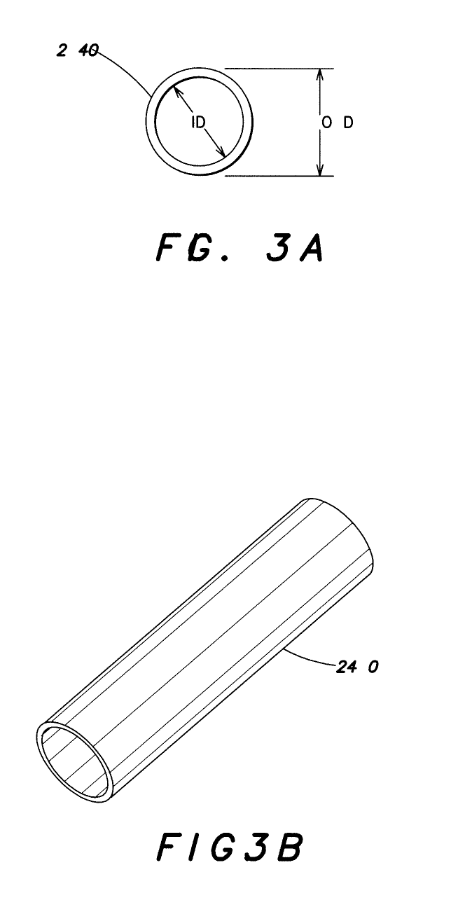

FIGS. 3A and 3B illustrate plan and projection views of sleeve 240. In the illustrated example embodiment, sleeve 240 is a simple cylindrically-shaped tube, having an inner diameter ID selected to provide a selected amount of friction between the plunger and the inner surface of the sleeve as the plunger moves slidingly along the inner surface of the sleeve.

Similarly, in the illustrated embodiment, sleeve 240 has an outer diameter OD selected to provide a selected amount of friction between the lumen wall and the outer surface of the sleeve as the sleeve moves slidingly along inner surface of the lumen wall. The relative amount of friction between the plunger and the sleeve, and between the sleeve and the lumen wall determines the amount of relative movement between the sleeve and the injector body for a given amount of relative movement between the plunger and the injector body as the plunger is moved to inject an IOL.

For example, in some embodiments the friction between the plunger and the sleeve and the friction between the sleeve and the lumen wall are chosen to be about equal, and the stroke S′ is selected to be about twice as long as the distance between the distal surface 235 of plunger body and the distal end 242 of the sleeve, at full retraction; and twice as long as the distance between the distal end 242 of the sleeve and surface 225, at full retraction. As a result, when the thumb press 236 is pressed, the plunger will reach the end of its stroke when the plunger body reaches the end 242 of sleeve 240, and when surface 235 reaches surface 225. The dimensions and friction values are given by way of illustration and any suitable dimensions, and any suitable stroke lengths and friction values may be used.

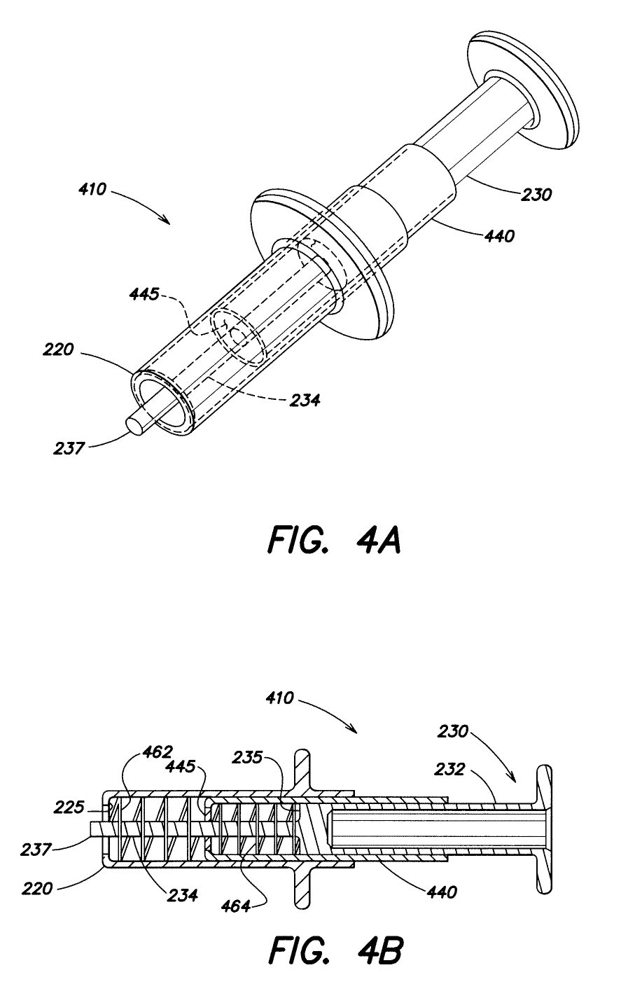

FIG. 4A is a schematic illustration of another example of an injector 410 according to aspects of the invention including an injector body 220, a plunger 230 and a sleeve 440. The injector body and the plunger are substantially similar to those in the embodiment of FIG. 2A. Sleeve 440 is also similar to the sleeve in FIG. 2A, except an end cap 445 is provided on the sleeve to support the plunger shaft 234, which thereby reduces the distance over which the plunger tip 237 is cantilevered.

In some embodiments (as shown in FIG. 4B), an amount of movement between the sleeve and the injector body, for a given amount of movement between the sleeve and the plunger may be achieved using optional springs 462, 464. Spring 462 is disposed between sleeve 440 (e.g., end cap 445) and injector body 220 (e.g., surface 225); and spring 464 is disposed between plunger body 232 (e.g., surface 235) and sleeve 440 (e.g.,. end cap 445). The spring constants of the springs may be selected to achieve appropriate relative movements of the injector body, the plunger and the sleeve. In such embodiments, the friction between the sleeve and the injector body, and the friction between the plunger and the sleeve may be less important than when the springs are omitted.

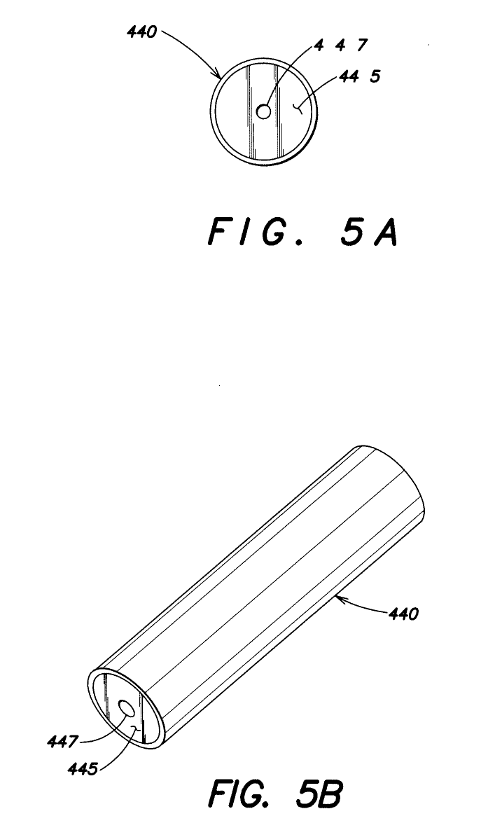

FIGS. 5A and 5B illustrate plan and projection views of sleeve 440, respectively. A hole 447 in the end cap 445 is selected to have a size and shape that corresponds to the size and shape of the plunger shaft 234 (shown in FIG. 4A). Any suitable amount of friction may occur between the boundaries of the hole and the shaft of the plunger, provided that the end cap provides suitable support for the plunger shaft and plunger tip. The hole may form a continuous circle that contacts the shaft, or one or more discontinuous portions that contact the shaft at selected locations about its circumference.

It will be appreciated that, in some embodiments where a sleeve has an end cap, the sleeve provides support for the body of the plunger, and the end cap with a hole provides support for the shaft of the plunger.

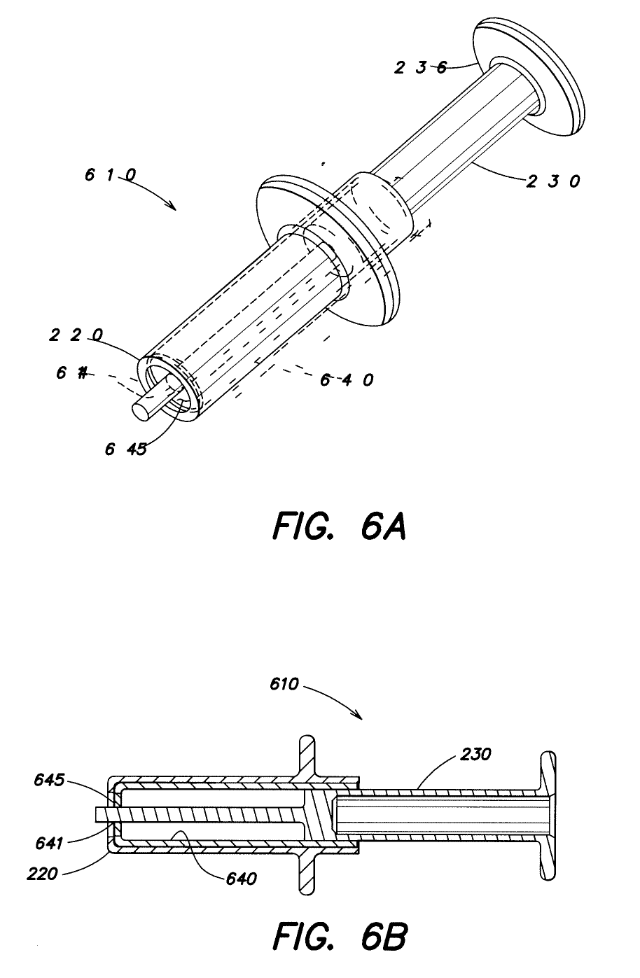

FIG. 6A is a schematic illustration of another embodiment of an injector 610 according to aspects of the invention including an injector body 220, a plunger 230 and a sleeve 640. The injector body and the plunger are substantially similar to those in the embodiment of FIG. 2A.

In injector 610, sleeve 640 is maintained in a fixed location within the injector body. The sleeve may be maintained in a fixed location by an interference fit, application of an adhesive, a fastener or any other suitable technique.

The sleeve may extend further in a proximal direction (i.e., toward the thumb press 236) than the injector body to increase the contact distance with the plunger relative the contact distance that would exist with the injector body in the absence of the sleeve; however, a fixed sleeve need not be so configured. Accordingly, in some embodiments, the contact distance between the plunger and the sleeve may be greater than, the same or less than would exist between the plunger and the injector body in the absence of the sleeve. It will be appreciated that, regardless of the contact distance between the plunger and the injector body, the end cap 645 with a hole 641 provides support that decreases the cantilever of the tip.

An additional advantage of providing a fixed sleeve (or a slidable sleeve) includes the ability to introduce a sleeve of a different material than the injector body. For example, in some embodiments, it may be desirable that the injector body and plunger be made of material or materials having certain structural strengths, but such materials may have inappropriate sliding characteristics when in contact with one another. Accordingly a fixed sleeve having a more suitable sliding characteristic with the plunger may be used. For example, the injector body and the plunger may be a rigid polymer material or metal, and the sleeve may be polypropylene. A sleeve having a different material than at least one of the plunger and the injector body may be used in embodiments where the sleeve is maintained in a fixed location within the injector body, or where the sleeve is slidable relative to injector body.

Having thus described the inventive concepts and a number of exemplary embodiments, it will be apparent to those skilled in the art that the invention may be implemented in various ways, and that modifications and improvements will readily occur to such persons. Thus, the embodiments are not intended to be limiting and presented by way of example only. The invention is limited only as required by the following claims and equivalents thereto.

What is claimed is:

1. An IOL injector, comprising:
an injector body having a lumen wall defining a lumen;
a plunger configured and arranged to move through the lumen; and
a sleeve, the sleeve configured and arranged so as to contact at least a portion of the lumen wall and to slidably engage with at least a portion of the plunger.

2. The IOL injector of claim 1, wherein the sleeve is configured and arranged so as to slidably engage the lumen wall.

3. The IOL injector of claim 1, wherein the sleeve comprises a cylindrically-shaped tube.

4. The IOL injector of claim 1, wherein the sleeve comprises an end cap configured and arranged to support a shaft of the plunger.

5. The IOL injector of claim 1, further comprising a first spring disposed between the sleeve and the injector body, and a second spring disposed between the plunger body and the sleeve.

6. The IOL injector of claim 1, wherein the sleeve is adapted to be maintained in a fixed location within the injector body.

7. The IOL injector of claim 1, wherein the sleeve comprises a different material than at least one of the plunger and the injector body.

8. The IOL injector of claim 7, wherein the sleeve comprises a different material than the plunger.

9. The IOL injector of claim 7, wherein the sleeve comprises a different material than the injector body.
